(19) United States
(12) Patent Application Publication
(10) Pub. No.: US 2004/0210140 A1
(43) Pub. Date: Oct. 21, 2004

(54) Apparatus and method for preshaped ultrasonic probe

(75) Inventors: Robert A. Rabiner, North Reading, MA (US); Bradley A. Hare, Chelmsford, MA (US)

Correspondence Address: PALMER & DODGE, LLP, RICHARD B. SMITH, 111 HUNTINGTON AVENUE, BOSTON, MA, 02199, US

(73) Assignee: OmniSonics Medical Technologies, Inc.

(21) Appl. No.: 10/414,368

(22) Filed: Apr. 15, 2003

(51) Int. Cl. A61B 8/14

(52) U.S. Cl. 600/459

(57) ABSTRACT

The present invention provides an apparatus and a method for using a preshaped ultrasonic probe to ablate an occlusion in a vasculature. An ultrasonic medical device comprising an ultrasonic probe having a proximal end, a distal end and a longitudinal axis therebetween and a preshaped segment along the longitudinal axis increases a surface area of the ultrasonic probe in communication with the occlusion. A method of increasing a treatment area of an ultrasonic probe to ablate an occlusion comprising inserting the ultrasonic probe with a preshaped segment into a catheter, advancing the preshaped segment beyond a distal end of the catheter and activating an ultrasonic energy source to provide an ultrasonic energy to the ultrasonic probe to ablate the occlusion. The present invention increases the surface area in communication with the occlusion, maximizes a radial span in a vasculature and expands a treatment area of the ultrasonic probe.

### RELATED APPLICATION(S)

None.

### FIELD OF THE INVENTION

The present invention relates to an ultrasonic medical device, and more particularly to an apparatus and a method for an ultrasonic probe having a preshaped segment along a longitudinal axis that increases a surface area of the ultrasonic probe in communication with an occlusion to ablate the occlusion in a vasculature of a body.

### BACKGROUND OF THE INVENTION

Vascular occlusive disease affects millions of individuals worldwide and is characterized by a dangerous blockage of blood vessels. Vascular occlusive disease includes thrombosed hemodialysis grafts, peripheral artery disease, deep vein thrombosis, coronary artery disease, heart attack and stroke. Vascular occlusions (including, but not limited to, clots, intravascular blood clots or thrombus, occlusional deposits, such as calcium deposits, fatty deposits, atherosclerotic plaque, cholesterol buildup, fibrous material buildup and arterial stenoses) result in the restriction or blockage of blood flow in the vessels in which they occur. Occlusions result in oxygen deprivation (“ischemia”) of tissues supplied by these blood vessels. Prolonged ischemia results in permanent damage of tissues which can lead to limb loss, myocardial infarction, stroke, or death. Targets for occlusion include coronary arteries, peripheral arteries and other blood vessels.

The disruption of an occlusion can be affected by pharmacological agents, mechanical methods, ultrasonic methods or combinations of all three. Many procedures involve inserting a medical device into a vasculature of the body. Medical devices include, but are not limited to, probes, catheters, wires, tubes and similar devices. In some cases, the medical device delivers a pharmacological agent to the site of the occlusion.

Navigation of a probe within a vasculature of a body to a site of an occlusion can be a challenging process for a surgeon. The difficulty of the navigation lies in the path of the particular vasculature that is being navigated, the degree of blockage of the occlusion of biological material and the physical properties of the probe. Probes need to have a degree of rigidity in order for a surgeon to be able to control the insertion process through the tortuous paths of the vasculature. Often times, a torque is applied to the probe to move the probe through the vasculature. In addition, probes need to have a degree of flexibility so the probe can flex, bend and curve according to the path of the vasculature. The flexibility also reduces the potential risk of damage to the healthy tissue as the probe is being navigated within the vasculature.

The use of a large diameter probe within the vasculature of the body has been proposed, but has several disadvantages. Navigation of the probe through the vasculatures of the body is difficult due to the high stresses that are required to bend the probe as the user applies force and/or torque to move the probe to the treatment site. As the diameter of the probe increases, it is more difficult to bend the probe. Applications where the probe is used in a vasculature deep within the body present the largest challenge for the user. The high stresses that are imparted to the walls of the vasculature in the body as the probe is moved to the treatment site can weaken the vasculature. Often times, the probe is moved to the treatment site after a series of probe withdrawals and probe re-insertions, with each withdrawal and insertion of the probe potentially weakening the vasculature. In order to alleviate these problems, it is desirable that the geometry of the distal end of the probe match the anatomy of the vasculature. In addition, there is a need in the art for a probe that increases a surface area of the probe in communication with an occlusion.

In addition to the weakening of the vasculature and the need to reduce stresses, ultrasonic probes having a large diameter require a higher amount of power in order to vibrate them. It is desirable to minimize the power during the ultrasonic vibration of the probe, since increased power levels lead to excess heating of the probe, potential damage to the vasculature and the patient, and functional limitations of the probe. Straight probes used in the ablation of an occluded material also require a high power output to maximize the effect of the ultrasonic energy and require long treatment times for the ablation of the occluded material. Therefore, there is a need in the art for an ultrasonic probe that increases the surface area in communication with the occlusion so the required power to ablate the occlusion can be minimized to eliminate potential damage to the vasculature and the patient.

The prior art has not addressed the problem of increasing the surface area of an ultrasonic probe in communication with an occlusion to ablate the occlusion. U.S. Pat. No. 6,099,464 to Shimizu et al. discloses a bending sheath for a probe where the tip portion of the probe is bent by forcibly inserting the probe into the bending sheath. The amount that the probe is bent is controlled by the length that is inserted into the bending sheath. The Shimizu et al. device is a complicated system that imparts high stresses on the probe as the probe is forcibly bent as it is moved into the bending sheath, thereby compromising the functionality of the probe by altering the frequency of vibration of the probe and initiating high stress concentration sites along the axis of the probe. In addition, the Shimizu et al. device has a large diameter that could not be used in a vasculature deep within the body, would require a long treatment time and would require high power that could damage healthy tissue. Therefore, there is a need in the art for an apparatus and method of delivering a preshaped ultrasonic probe to a site of an occlusion within a vasculature that reduces the treatment time to ablate the occlusion, preserves the ultrasonic properties of the ultrasonic probe, does not compromise the structural integrity of the ultrasonic probe, matches the anatomy of the vasculature, does not harm the patient or the vasculature the ultrasonic probe is moving through, can be delivered within a vasculature deep within the body to ablate the occlusion and increases the surface area of the ultrasonic probe in communication with the occlusion.

U.S. Pat. No. 5,910,129 to Koblish et al. discloses a catheter assembly having a sheath, a catheter tube, a pull wire and a multiple electrode structure with a plurality of electrode elements at the distal end of the catheter assembly. The user deploys a looped structure by advancing the catheter tube through the sheath and pulling on the pull wire that is positioned within the sheath. The looped structure allows for a degree of contact between the tissue and the electrode elements, with the electrode elements transmitting electromagnetic radio frequency energy to ablate tissue. The Koblish et al. device is a bulky device that comprises a large amount of parts and elements that interact to help navigate the Koblish et al. device to the treatment site. The electrode elements of the multiple electrode structure in the Koblish et al. device make the surface along the distal end of the longitudinal axis of the Koblish et al. device irregular, rough and bulky, thereby imparting high stresses to the vasculature, potentially rupturing the vasculature, potentially damaging healthy tissue and providing non-uniform energy transfer for ablation of tissue. Therefore, there remains a need in the art for an apparatus and method of delivering a preshaped ultrasonic probe to a site of an occlusion within a vasculature that reduces the treatment time to ablate the occlusion, preserves the ultrasonic properties of the ultrasonic probe, does not compromise the structural integrity of the ultrasonic probe, matches the anatomy of the vasculature, does not harm the patient or the vasculature the ultrasonic probe is moving through, can be delivered within a vasculature deep within the body to ablate the occlusion and increases the surface area of the ultrasonic probe in communication with the occlusion.

U.S. Pat. No. 6,512,957 to Witte discloses a device for the introduction into a blood vessel that includes a catheter with a plurality of tines and an at least one ring of electrodes along the surface of the catheter, a formed wire and an exit lock mechanism. The catheter is advanced through the blood vessel, with the formed wire inside of the catheter, and at a branch of the blood vessel, the formed wire is advanced past the tip of the catheter with the shape of the formed wire allowing for negotiation through the blood vessel. The Witte device comprises numerous complex components that limits the vasculatures the Witte device can be used in and the plurality of tines and at least one ring of electrodes impart high stresses to the walls of the vessel that could damage the vessel. In addition, the Witte device has a formed shape that could damage the vessel after advancement past the tip of the catheter. Therefore, there is a need in the art for an apparatus and method of delivering a preshaped ultrasonic probe to a site of an occlusion within a vasculature that reduces the treatment time to ablate the occlusion, preserves the ultrasonic properties of the ultrasonic probe, does not compromise the structural integrity of the ultrasonic probe, matches the anatomy of the vasculature, does not harm the patient or the vasculature the ultrasonic probe is moving through, can be delivered within a vasculature deep within the body to ablate the occlusion and increases the surface area of the ultrasonic probe in communication with the occlusion.

The prior art devices do not solve the problem of providing an ultrasonic probe that increases an active area between an occlusion and the ultrasonic probe to ablate the occlusion. The prior art devices do not solve the problem of delivering a probe within a vasculature to ablate an occlusion of a biological material without imparting high stresses to the vasculatures and potentially damaging healthy tissue. The prior art devices can not be used in vasculatures deep within the body and the prior art devices compromise the ultrasonic performance of the devices. The prior art devices require a high amount of power that can damage the vasculatures and require long treatment times that can adversely affect healthy tissue in the patient. Therefore, there remains a need in the art for an apparatus and method of delivering an ultrasonic probe to a site of an occlusion within a vasculature that reduces the treatment time to ablate the occlusion, preserves the ultrasonic properties of the ultrasonic probe, does not compromise the structural integrity of the ultrasonic probe, matches the anatomy of the vasculature, does not harm the patient or the vasculature the ultrasonic probe is moving through, can be delivered within a vasculature deep within the body to ablate the occlusion and increases the surface area of the ultrasonic probe in communication with the occlusion.

### SUMMARY OF THE INVENTION

The present invention relates to an ultrasonic medical device, and more particularly to an apparatus and a method for an ultrasonic probe having a preshaped segment along a longitudinal axis that increases a surface area of the ultrasonic probe in communication with an occlusion to ablate the occlusion in a vasculature of a body.

The present invention is an ultrasonic medical device comprising an ultrasonic probe having a proximal end, a distal end and a longitudinal axis therebetween, and a preshaped segment along the longitudinal axis wherein the preshaped segment increases a surface area of the ultrasonic probe in communication with a biological material. In a preferred embodiment of the present invention, the preshaped segment is located at a distal end of the ultrasonic probe.

The present invention is an ultrasonic medical device for ablating an occlusion comprising an elongated flexible probe having a preshaped segment along a longitudinal axis that maximizes a radial span of the elongated flexible probe within a vasculature of a body. A catheter surrounds a length of the longitudinal axis of the elongated flexible probe. The elongated flexible probe supports a transverse ultrasonic vibration along a portion of the longitudinal axis of the elongated flexible probe to ablate the occlusion. The preshaped segment of the elongated flexible probe focuses a delivery of a transverse ultrasonic energy to the occlusion.

The present invention provides a method of expanding a treatment area of an ultrasonic probe to ablate a biological material in a vasculature of a body by inserting the ultrasonic probe having a preshaped segment along a longitudinal axis into a catheter, advancing the preshaped segment beyond a distal end of the catheter and activating an ultrasonic energy source to provide an ultrasonic energy to the ultrasonic probe to ablate the biological material. The preshaped segment engages the biological material for ablation.

The present invention provides a method of increasing a surface area of a flexible ultrasonic probe in communication with an occlusion. The flexible ultrasonic probe with a preshaped segment along a longitudinal axis is advanced to a site of the occlusion and the preshaped segment is moved in communication to the occlusion. An ultrasonic energy source is activated to vibrate the longitudinal axis of the flexible ultrasonic probe in a transverse direction to ablate the occlusion.

The present invention is an ultrasonic medical device comprising an ultrasonic probe with a preshaped segment along a longitudinal axis. The preshaped segment of the ultrasonic probe increases the surface area of the ultrasonic probe in communication with an occlusion and maximizes a radial span of the ultrasonic probe in a vasculature to remove the occlusion. The present invention provides a presbaped ultrasonic probe that is safe, simple, user-friendly, reliable and cost effective.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 shows a side plan view of an ultrasonic medical device of the present invention capable of operating in a transverse mode comprising an ultrasonic probe with a preshaped segment along a longitudinal axis that is S shaped.

FIG. 2 shows a fragmentary side plan view of a section of a longitudinal axis of the ultrasonic probe including a preshaped segment moved past a distal end of a catheter and engaging an occlusion within a vasculature of a body.

FIG. 3 shows a side plan view of an alternative embodiment of an ultrasonic medical device of the present invention showing a plurality of transverse nodes and a plurality of transverse anti-nodes along a portion of a longitudinal axis of the ultrasonic probe having a uniform diameter.

FIG. 4 shows a side plan view of an ultrasonic medical device of the present invention capable of operating in a transverse mode comprising an ultrasonic probe with a preshaped segment along a longitudinal axis at an angle to the longitudinal axis of the ultrasonic probe.

FIG. 5 shows a side plan view of an ultrasonic medical device of the present invention capable of operating in a transverse mode comprising an ultrasonic probe with a preshaped segment along a longitudinal axis that is sinusoidal shaped.

FIG. 6 shows a side plan view of an ultrasonic medical device of the present invention capable of operating in a transverse mode comprising an ultrasonic probe with a preshaped segment along a longitudinal axis that is hook shaped.

FIG. 7 shows a fragmentary perspective view of the ultrasonic probe of the present invention with a preshaped segment along a longitudinal axis that is corkscrew shaped.

FIG. 8 shows a fragmentary perspective view of the ultrasonic probe of the present invention with a preshaped segment along a longitudinal axis that is coil shaped similar to a spring.

FIG. 9 shows a side plan view of an ultrasonic medical device of the present invention capable of operating in a transverse mode comprising an ultrasonic probe with a preshaped segment along a longitudinal axis that is curved.

FIG. 10 shows a side plan view of an ultrasonic medical device of the present invention capable of operating in a transverse mode comprising an ultrasonic probe with a preshaped segment along a longitudinal axis that is S shaped and located between a proximal end and a distal end of the ultrasonic probe.

FIG. 11 shows a side plan view of an ultrasonic medical device of the present invention capable of operating in a transverse mode comprising an ultrasonic probe with a hook preshaped segment 43 located at a distal end 24 of the ultrasonic probe 15 and an S-shaped preshaped segment 43 between the proximal end 31 and the distal end 24 of the ultrasonic probe 15.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and spirit of the principles of the present invention.

### DETAILED DESCRIPTION

The present invention provides an apparatus and a method for using a preshaped ultrasonic probe. An ultrasonic medical device comprises an ultrasonic probe having a preshaped segment along a longitudinal axis. In a preferred embodiment of the present invention, the preshaped segment of the ultrasonic probe is located at a distal end of the ultrasonic probe. In a preferred embodiment of the present invention, a catheter surrounds a length of the longitudinal axis of the ultrasonic probe. In a preferred embodiment of the present invention, the ultrasonic probe and the catheter are moved to a site of an occlusion and a section of the longitudinal axis of the ultrasonic probe is advanced past a distal end of the catheter and an ultrasonic energy source is activated. The preshaped segment of the ultrasonic probe increases the surface area of the ultrasonic probe in communication with the occlusion to ablate the occlusion. The preshaped segment of the ultrasonic probe maximizes a radial span of the ultrasonic probe within the vasculature. The preshaped segment of the ultrasonic probe adapts to a contour of a vasculature in a body and expands a treatment area of the ultrasonic probe.

The following terms and definitions are used herein:

“Ablate” as used herein refers to removing, clearing, destroying or taking away a biological material. “Ablation” as used herein refers to a removal, clearance, destruction, or taking away of the biological material.

“Node” as used herein refers to a region of a minimum energy emitted by an ultrasonic probe at or proximal to a specific location along a longitudinal axis of the ultrasonic probe.

“Anti-node” as used herein refers to a region of a maximum energy emitted by an ultrasonic probe at or proximal to a specific location along a longitudinal axis of the ultrasonic probe.

“Probe” as used herein refers to a device capable of propagating an energy emitted by the ultrasonic energy source along a longitudinal axis of the probe, resolving the energy into an effective cavitational energy at a specific resonance (defined by a plurality of nodes and a plurality of anti-nodes along an “active area” of the probe) and is capable of an acoustic impedance transformation of electrical energy to a mechanical energy.

“Transverse” as used herein refers to a vibration of a probe not parallel to a longitudinal axis of the probe. A “transverse wave” as used herein is a wave propagated along the probe in which a direction of a disturbance at a plurality of points of a medium is not parallel to a wave vector.

“Biological material” as used herein refers to a collection of a matter including, but not limited to, a group of similar cells, intravascular blood clots or thrombus, fibrin, calcified plaque, calcium deposits, occlusional deposits, atherosclerotic plaque, fatty deposits, adipose tissues, atherosclerotic cholesterol buildup, fibrous material buildup, arterial stenoses, minerals, high water content tissues, platelets, cellular debris, wastes and other occlusive materials.

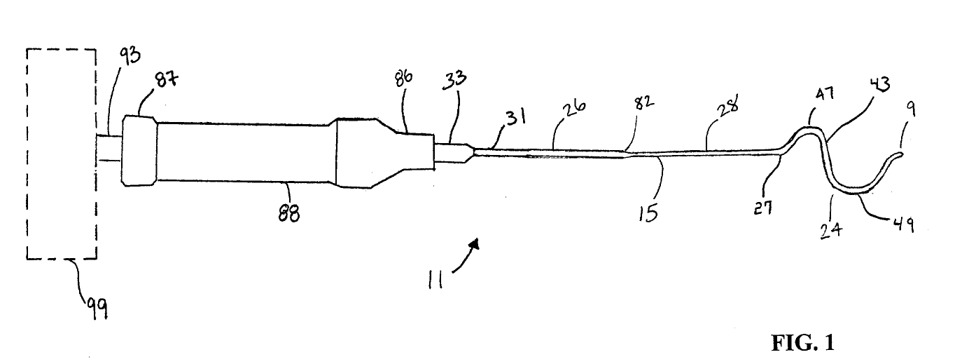

FIG. 1 shows an ultrasonic medical device 11 of the present invention. The ultrasonic medical device 11 includes an ultrasonic probe 15 which is coupled to an ultrasonic energy source or generator 99 (shown in phantom in FIGS. 1, 3-6 and 9-10) for the production of an ultrasonic energy. A handle 88, comprising a proximal end 87 and a distal end 86, surrounds a transducer within the handle 88. The transducer, having a first end engaging the ultrasonic energy source 99 and a second end engaging a proximal end 31 of the ultrasonic probe 15, transmits the ultrasonic energy to the ultrasonic probe 15. A connector 93 engages the ultrasonic energy source 99 to the transducer. The ultrasonic probe 15 includes the proximal end 31 and a distal end 24 that ends in a probe tip 9. The ultrasonic probe 15 has a preshaped segment 43 along a longitudinal axis that comprises a first outermost point 47 and a second outermost point 49 at a maximum displacement from the longitudinal axis of the ultrasonic probe 15. The ultrasonic probe 15 has a preshape transition 27 that identifies a deviation from the straight portion of the longitudinal axis and the start of the preshaped segment 43 of the ultrasonic probe 15. A diameter of the ultrasonic probe 15 decreases from a first interval 26 to a second interval 28 along the longitudinal axis of the ultrasonic probe 15 over an at least one diameter transition 82. A quick attachment-detachment system 33 that engages the proximal end 31 of the ultrasonic probe 15 to the transducer within the handle 88 is illustrated generally in FIGS. 1, 3-6 and 9-10. An ultrasonic probe device with a quick attachment-detachment system is described in the Assignee's co-pending patent applications U.S. Ser. No. 09/975,725; U.S. Ser. No. 10/268,487; U.S. Ser. No. 10/268,843, and the entirety of all these applications are hereby incorporated herein by reference.

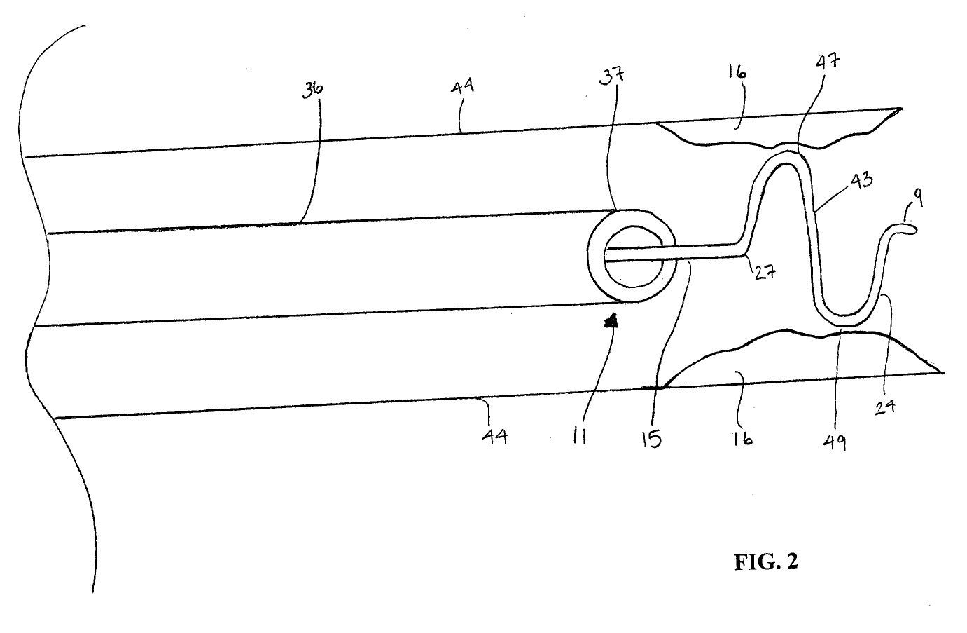

FIG. 2 shows a fragmentary view of the longitudinal axis of the ultrasonic probe 15 having the preshaped segment 43 extended beyond a distal end 37 of a catheter 36 and engaging an occlusion 16 inside a vasculature 44. The preshaped segment 43 of the ultrasonic probe 15 increases a surface area of the ultrasonic probe 15 in communication with the occlusion 16 and maximizes a radial span of the ultrasonic probe 15 within the vasculature 44.

The handle 88 surrounds the transducer located between the proximal end 31 of the ultrasonic probe 15 and the connector 93. In a preferred embodiment of the present invention, the transducer includes, but is not limited to, a horn, an electrode, an insulator, a backnut, a washer, a piezoelectric microphone, and a piezoelectric drive. The transducer converts electrical energy provided by the ultrasonic energy source 99 to mechanical energy. The transducer transmits the ultrasonic energy received from the ultrasonic energy source 99 to the ultrasonic probe 15. Energy from the ultrasonic energy source 99 is transmitted along the longitudinal axis of the ultrasonic probe 15, causing the ultrasonic probe 15 to vibrate in a transverse mode. The transducer is capable of engaging the ultrasonic probe 15 at the proximal end 31 with sufficient restraint to form an acoustical mass that can propagate the ultrasonic energy provided by the ultrasonic energy source 99.

In a preferred embodiment of the present invention, the transducer transmits the ultrasonic energy from the ultrasonic energy source 99 to the longitudinal axis of the ultrasonic probe 15 to oscillate the ultrasonic probe 15 in a direction transverse to its longitudinal axis. In a preferred embodiment of the present invention, the transducer is a piezoelectric transducer that is coupled to the ultrasonic probe 15 to enable transfer of ultrasonic excitation energy and cause the ultrasonic probe 15 to oscillate in the transverse direction relative to the longitudinal axis. In an alternative embodiment of the present invention, a magneto-strictive transducer may be used for transmission of the ultrasonic energy.

The ultrasonic probe 15 comprises the preshaped segment 43 along a longitudinal axis that increases the surface area in communication with the occlusion 16 and matches an anatomy of the vasculature 44. In a preferred embodiment of the present invention, the occlusion 16 comprises a biological material. In a preferred embodiment of the present invention, the preshaped segment 43 is located at the distal end 24 of the ultrasonic probe 15. In a preferred embodiment of the present invention, the ultrasonic probe 15 ends in the probe tip 9. The probe tip 9 can be any shape including, but not limited to, bent, a ball or larger shapes. In an embodiment of the present invention, the ultrasonic energy source 99 is a physical part of the ultrasonic medical device 11. In another embodiment of the present invention, the ultrasonic energy source 99 is not a physical part of the ultrasonic medical device 11.

In an embodiment of the present invention shown in FIG. 1, the diameter of the ultrasonic probe 15 decreases from the first interval 26 to the second interval 28. In another embodiment of the present invention, the diameter of the ultrasonic probe 15 decreases at greater than two intervals. In a preferred embodiment of the present invention, the diameter transitions 82 of the ultrasonic probe 15 are tapered in a gradual manner to change the diameter from the proximal end 31 to the distal end 24 along the longitudinal axis of the ultrasonic probe 15. In another embodiment of the present invention, the diameter transitions 82 of the ultrasonic probe 15 are stepwise to change the diameter from the proximal end 31 to the distal end 24 along the longitudinal axis of the ultrasonic probe 15. Those skilled in the art will recognize that there can be any number of intervals and diameter transitions and that the diameter transitions can be of any shape known in the art and be within the spirit and scope of the present invention.

In a preferred embodiment of the present invention, the ultrasonic probe 15 has a small diameter. In a preferred embodiment of the present invention, the ultrasonic probe 15 is a wire. In a preferred embodiment of the present invention, the diameter of the ultrasonic probe 15 decreases in a gradual manner from the proximal end 31 to the distal end 24. In an embodiment of the present invention, the diameter of the distal end 24 of the ultrasonic probe 15 is about 0.004 inches. In another embodiment of the present invention, the diameter of the distal end 24 of the ultrasonic probe 15 is about 0.015 inches. In other embodiments of the present invention, the diameter of the distal end 24 of the ultrasonic probe 15 varies between about 0.003 inches and about 0.025 inches. Those skilled in the art will recognize an ultrasonic probe 15 can have a diameter at the distal end 24 smaller than about 0.003 inches, larger than about 0.025 inches, and between about 0.003 inches and about 0.025 inches and be within the spirit and scope of the present invention.

In an embodiment of the present invention, the diameter of the proximal end 31 of the ultrasonic probe 15 is about 0.012 inches. In another embodiment of the present invention, the diameter of the proximal end 31 of the ultrasonic probe 15 is about 0.025 inches. In other embodiments of the present invention, the diameter of the proximal end 31 of the ultrasonic probe 15 varies between about 0.003 inches and about 0.025 inches. Those skilled in the art will recognize the ultrasonic probe 15 can have a diameter at the proximal end 31 smaller than about 0.003 inches, larger than about 0.025 inches, and between about 0.003 inches and about 0.025 inches and be within the spirit and scope of the present invention.

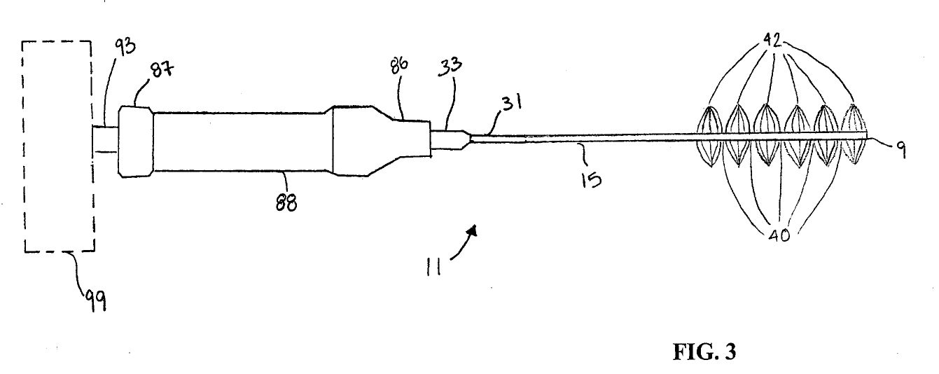

In an embodiment of the present invention shown in FIG. 3, the diameter of the ultrasonic probe 15 is approximately uniform from the proximal end 31 to the distal end 24 of the ultrasonic probe 15. In another embodiment of the present invention, the diameter of the ultrasonic probe 15 decreases in a gradual manner from the proximal end 31 to the distal end 24. In an embodiment of the present invention, the ultrasonic probe 15 resembles a wire. In an embodiment of the present invention, the gradual change of the diameter from the proximal end 31 to the distal end 24 occurs over the at least one diameter transitions 82 with each diameter transition 82 having an approximately equal length. In another embodiment of the present invention, the gradual change of the diameter from the proximal end 31 to the distal end 24 occurs over a plurality of diameter transitions 82 with each diameter transition 82 having a varying length. The diameter transition 82 refers to a section where the diameter varies from a first diameter to a second diameter.

The ultrasonic probe 15 comprises an at least one preshape transition 27 along the longitudinal axis of the ultrasonic probe 15. The preshape transition 27 is a point along the longitudinal axis of the ultrasonic probe 15 where the longitudinal axis deviates from a straight length. Those skilled in the art will recognize there can be any number of preshape transitions along the longitudinal axis of the ultrasonic probe and the preshape transitions can be located at any point along the longitudinal axis of the ultrasonic probe and be within the spirit and scope of the present invention.

The length of the ultrasonic probe 15 of the present invention is chosen so as to be resonant in a transverse mode. In an embodiment of the present invention, the ultrasonic probe 15 is between about 30 centimeters and about 300 centimeters in length. In an embodiment of the present invention, the ultrasonic probe 15 is a wire. Those skilled in the art will recognize an ultrasonic probe can have a length shorter than about 30 centimeters, a length between about 30 centimeters and about 300 centimeters, and a length longer than about 300 centimeters and be within the spirit and scope of the present invention.

In a preferred embodiment of the present invention, a cross section of the ultrasonic probe 15 is circular. In other embodiments of the present invention, a shape of the cross section of the ultrasonic probe 15 includes, but is not limited to, square, trapezoidal, oval, triangular, circular with a flat spot and similar cross sections. Those skilled in the art will recognize that other cross sectional geometric configurations known in the art would be within the spirit and scope of the present invention.

The ultrasonic probe 15 is designed to have the cross section with a small profile, which also allows the ultrasonic probe 15 to flex along its length, thereby allowing the ultrasonic probe 15 to be used in a minimally invasive manner. The ultrasonic probe 15 has a stiffness that gives the ultrasonic probe 15 a flexibility so it can be articulated in the vasculature 44 of the body. A significant feature of the present invention resulting from the transversely generated energy is the retrograde movement of biological material, e.g., away from the probe tip 9 and along the longitudinal axis of the ultrasonic probe 15.

The transverse mode of vibration of the ultrasonic probe 15 according to the present invention differs from an axial (or longitudinal) mode of vibration disclosed in the prior art. Rather than vibrating in an axial direction, the ultrasonic probe 15 of the present invention vibrates in a direction transverse (not parallel) to the axial direction. As a consequence of the transverse vibration of the ultrasonic probe 15, the occlusion destroying effects of the ultrasonic medical device 11 are not limited to those regions of the ultrasonic probe 15 that may come into contact with the occlusion 16. Rather, as a section of the longitudinal axis of the ultrasonic probe 15 is positioned in proximity to the occlusion 16, a diseased area or lesion, the occlusion 16 is removed in all areas adjacent to a plurality of energetic transverse nodes and transverse anti-nodes that are produced along a portion of the longitudinal axis of the ultrasonic probe 15, typically in a region having a radius of up to about 6 mm around the ultrasonic probe 15. The preshaped segment 43 of the ultrasonic probe 15 allows for additional surface area coverage compared to a probe that is approximately straight along a longitudinal axis. The present invention allows a greater surface area of the preshaped segment 43 of the ultrasonic probe 15 to be positioned closer to the occlusion 16 than a probe that is approximately straight along the longitudinal axis. The present invention allows the preshaped segment 43 of the ultrasonic probe 15 to match the anatomy of the vasculature 44 and allows for a greater active area in communication with the occlusion 16 when compared to a probe that is approximately straight along the longitudinal axis. The present invention with the preshaped segment 43 allows for a lower amount of power in an ablation of the occlusion 16 when compared to a probe that is approximately straight along the longitudinal axis. The preshaped segment 43 is formed to move the ultrasonic probe 15 through the vasculature 44 of the body without damage to the vasculature 44.

Transversely vibrating ultrasonic probes for occlusion treatment are described in the Assignee's co-pending patent applications U.S. Ser. No. 09/776,015; U.S. Ser. No. 09/618,352; and U.S. Ser. No. 09/917,471, which further describe the design parameters for such an ultrasonic probe and its use in ultrasonic devices for a treatment, and the entirety of all these applications are hereby incorporated herein by reference.

FIG. 3 illustrates an alternative embodiment of the ultrasonic medical device 11 wherein the ultrasonic probe 15 has an approximately uniform diameter and is approximately straight along the longitudinal axis. The ultrasonic probe 15 comprises a plurality of transverse nodes 40 and transverse anti-nodes 42 at repeating intervals along a portion of the longitudinal axis of the ultrasonic probe 15. FIG. 3 generally indicates the location of the plurality of transverse nodes 40 and transverse anti-nodes 42 for the ultrasonic probe 15 that is approximately straight along the longitudinal axis.

The preshaped segment 43 of the ultrasonic probe 15 allows for a larger active area in communication with the occlusion 16 when compared to a probe that is approximately straight along the longitudinal axis. The preshaped segment 43 of the ultrasonic probe 15 engages the occlusion 16 and comprises a plurality of points along the longitudinal axis that are positioned closer to the occlusion 16 when compared to a probe that is approximately straight along the longitudinal axis. The preshaped segment 43 of the ultrasonic probe 15 maximizes a radial span of the ultrasonic probe 15 within the vasculature 44. The plurality of transverse nodes 40 and transverse anti-nodes 42 produced by the transverse ultrasonic vibration produces an occlusion destroying effect in a region around the longitudinal axis of the ultrasonic probe 15. The preshaped segment 43 of the ultrasonic probe 15 provides a plurality of points along the longitudinal axis that deviate from the approximately straight portion of the longitudinal axis originating from the proximal end 31 of the ultrasonic probe 15. Since the occlusion destroying effects are in a region having a radius of up to about 6 mm around the longitudinal axis of the ultrasonic probe 15, the preshaped segment 43 allows the occlusion destroying effects of the ultrasonic probe to cover a larger radial span of the vasculature 44 to ablate the occlusion 16.

The preshaped segment 43 of the ultrasonic probe 15 increases the surface area of the ultrasonic probe 15 in communication with the occlusion 16, expanding a treatment area of the ultrasonic probe 15. The preshaped segment 43 of the ultrasonic probe 15 allows the occlusion destroying effects to be focused on the occlusion 16, focusing a delivery of the transverse ultrasonic energy to the occlusion 16. The preshaped segment 43 of the ultrasonic probe 15 is adapted to engage the vasculature 44 and adapts to a contour of the vasculature 44. In addition, the preshaped segment 43 matches the anatomy of the vasculature 44 and is formed to not damage the vasculature 44.

The preshaped segment 43 of the ultrasonic probe 15 maximizes a radial span of the ultrasonic probe 15 and increases a surface area of the ultrasonic probe 15 in communication with the occlusion 16. Since the preshaped segment 43 of the ultrasonic probe 15 has the plurality of points along the longitudinal axis closer to the occlusion 16, the power required to vibrate the longitudinal axis of the ultrasonic probe 15 and ablate the occlusion 16 can be minimized. High power levels adversely affect the vasculature 44 and the patient. In addition, since the preshaped segment 43 of the ultrasonic probe 15 has the plurality of points along the longitudinal axis closer to the occlusion 16, the treatment time to remove the occlusion 16 is minimized. Long treatment times for the ablation of the occlusion 16 are undesirable as the vasculature 44 becomes more susceptible to potential damage the longer the ultrasonic probe 15 is inserted into the vasculature 44.

FIG. 1 illustrates the ultrasonic probe 15 having the preshaped segment 43 at the distal end 24 that is S shaped. There are several embodiments of the present invention with the preshaped segment 43 of the, ultrasonic probe 15 that are effective in increasing the surface area and the radial span in communication with the occlusion 16.

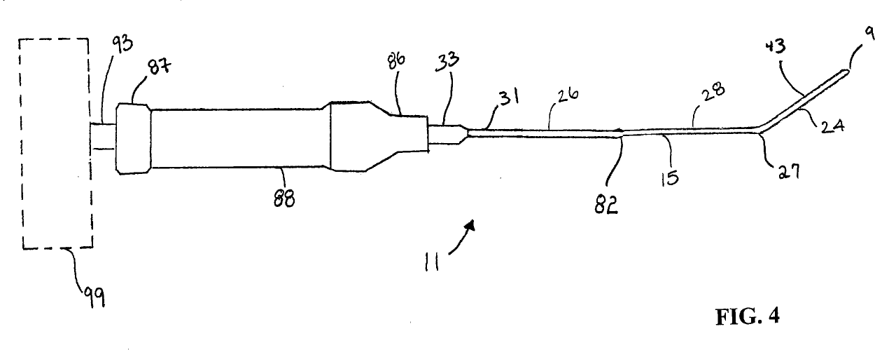

FIG. 4 shows another embodiment of the present invention in which the preshaped segment 43 of the ultrasonic probe 15 extends from the longitudinal axis of the ultrasonic probe 15 at an angle. The preshaped segment 43 of the ultrasonic probe 15 that extends from the longitudinal axis of the ultrasonic probe 15 is beneficial when the site of the occlusion 16 is proximal to a sharp bend in the vasculature 44.

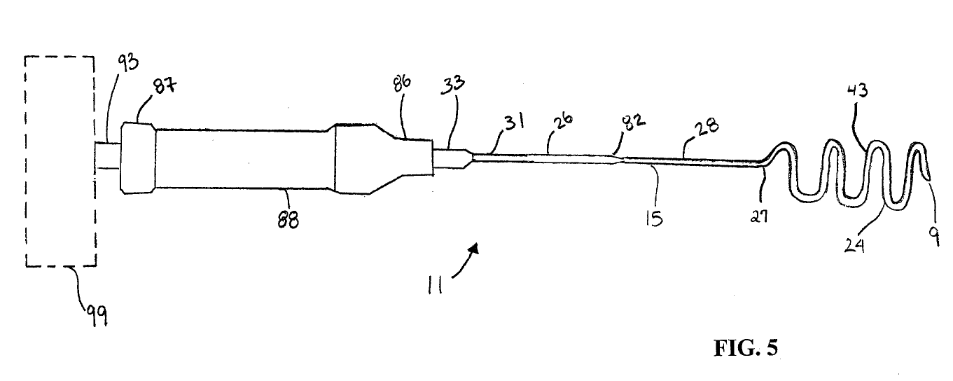

FIG. 5 shows another embodiment of the present invention in which the preshaped segment 43 of the ultrasonic probe 15 is sinusoidal shaped. The sinusoidal preshaped segment 43 of the ultrasonic probe 15 is beneficial when the occlusion 16 spans a long section of the vasculature 44 and provides the benefit of reducing the treatment time to ablate the occlusion.

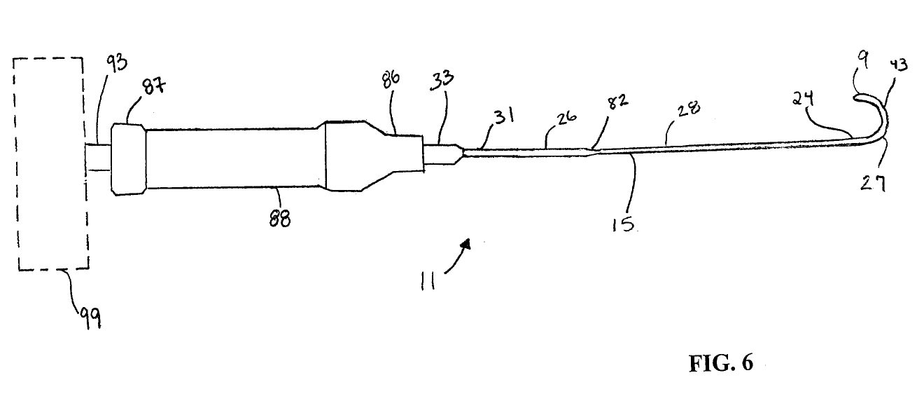

FIG. 6 shows another embodiment of the present invention in which the preshaped segment 43 of the ultrasonic probe 15 is hook shaped. The hook preshaped segment 43 of the ultrasonic probe 15 is beneficial when the site of the occlusion 16 is at a sharp bend in the vasculature 44.

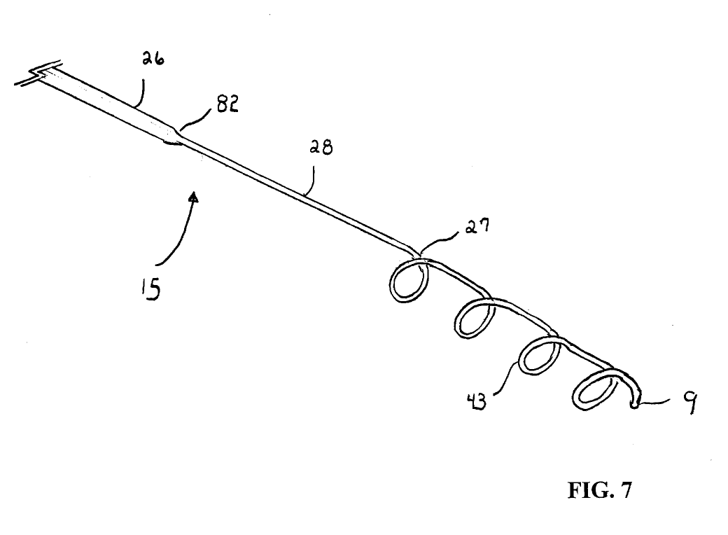

FIG. 7 shows a fragmentary perspective view of another embodiment of the present invention in which the preshaped segment 43 of the ultrasonic probe 15 resembles a corkscrew. The corkscrew preshaped segment 43 of the ultrasonic probe 15 is beneficial when the occlusion 16 spans a long section of the vasculature 44.

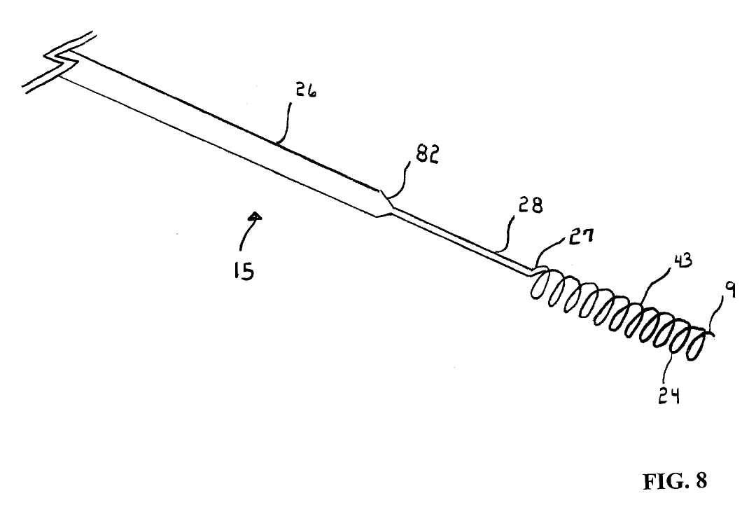

FIG. 8 shows a fragmentary perspective view of another embodiment of the present invention in which the preshaped segment 43 of the ultrasonic probe 15 resembles a coiled spring. The coiled spring preshaped segment 43 of the ultrasonic probe 15 is beneficial when the occlusion 16 spans a long section of the vasculature 44.

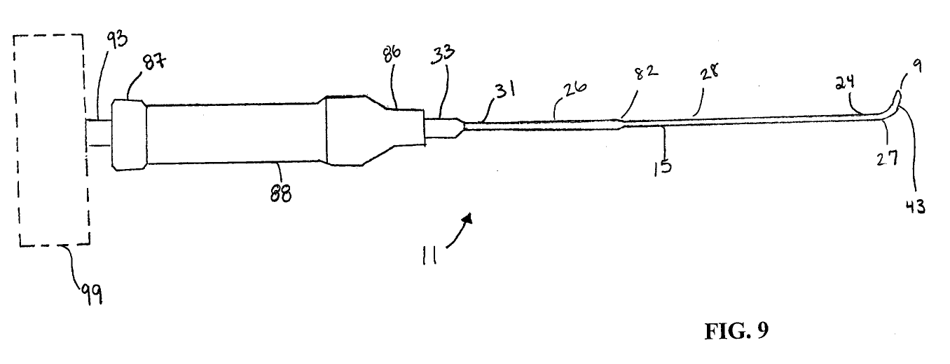

FIG. 9 shows another embodiment of the present invention in which the preshaped segment 43 of the ultrasonic probe 15 is curved. The curved preshaped segment 43 of the ultrasonic probe 15 is beneficial when the site of the occlusion 16 is proximal to a subtle bend in the vasculature 44. Those skilled in the art will recognize the preshaped segment 43 can be of many other shapes and geometric configurations and be within the spirit and scope of the present invention.

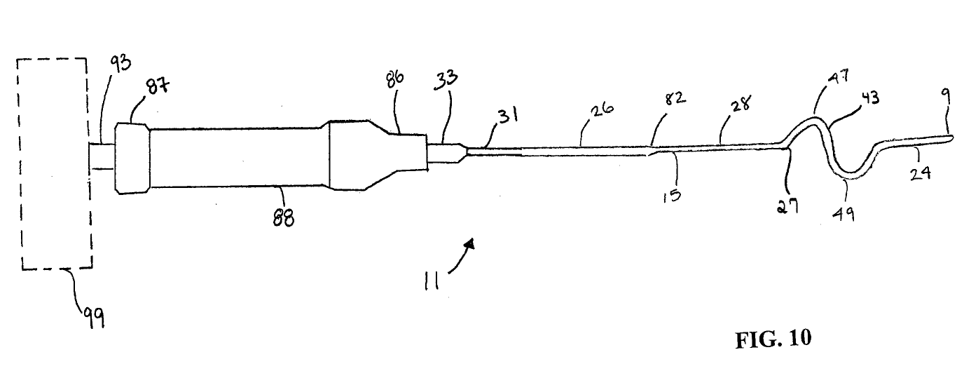

FIG. 10 shows another embodiment of the present invention in which the preshaped segment 43 of the ultrasonic probe 15 is S-shaped and located between the proximal end 31 and the distal end 24 of the ultrasonic probe 15. Those skilled in the art will recognize the preshaped segment can be located at any location along the longitudinal axis of the ultrasonic probe and be within the spirit and scope of the present invention.

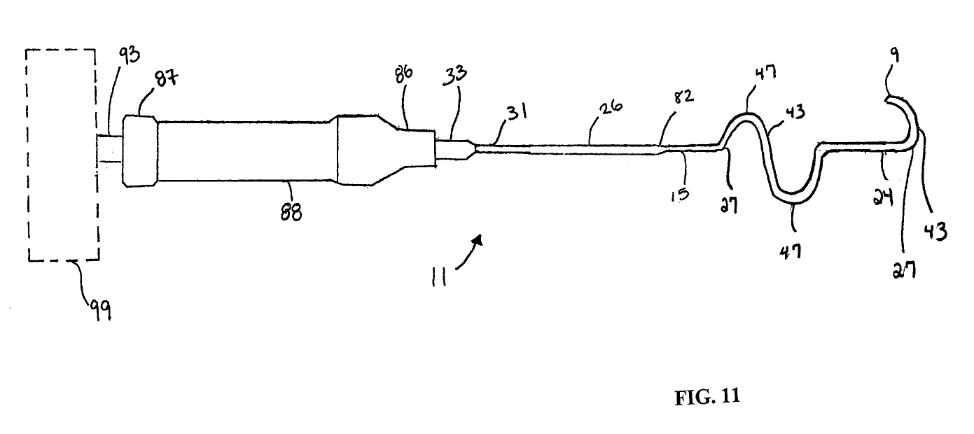

In a preferred embodiment of the present invention, there is a single preshaped segment 43 along the longitudinal axis of the ultrasonic probe 15. In alternative embodiments of the present invention, there can be more than one preshaped segment 43 along the longitudinal axis of the ultrasonic probe 15. For example, FIG. 11 shows an alternative embodiment of the present invention in which there are two preshaped segments 43 along the longitudinal axis of the ultrasonic probe 15. FIG. 11 illustrates an ultrasonic probe with a hook preshaped segment 43 located at a distal end of the ultrasonic probe 15 and an S-shaped preshaped segment 43 between the proximal end 31 and the distal end 24 of the ultrasonic probe 15. Those skilled in the art will recognize there can be any number of preshaped segments along the longitudinal axis of the ultrasonic probe and still be within the spirit and scope of the present invention.

Now referring to FIGS. 1-11, the length and the cross section of the ultrasonic probe 15 are sized to support the transverse ultrasonic vibration with the plurality of transverse nodes 40 and transverse anti-nodes 42 along the portion of the longitudinal axis of the ultrasonic probe 15. In a preferred embodiment of the present invention, more than one of the plurality of transverse anti-nodes 42 are in communication with the occlusion 16. The preshaped segment 43 of the ultrasonic probe 15 is adapted to engage the vasculature 44. The transverse ultrasonic vibration produces the plurality of transverse nodes 40 and transverse anti-nodes 42 along the portion of the longitudinal axis of the ultrasonic probe 15. The preshaped segment 43 of the present invention allows the plurality of transverse anti-nodes 42 with a high energy to be closer to the occlusion 16. The transverse nodes 40 are areas of a minimum energy and a minimum vibration. The plurality of transverse anti-nodes 42, or areas of a maximum energy and a maximum vibration, also occur at repeating intervals along the portion of the longitudinal axis of the ultrasonic probe 15. The number of transverse nodes 40 and transverse anti-nodes 42, and the spacing of the transverse nodes 40 and transverse anti-nodes 42 of the ultrasonic probe 15 depend on the frequency of the energy produced by the ultrasonic energy source 99. The separation of the transverse nodes 40 and the transverse anti-nodes 42 is a function of the frequency, and can be affected by tuning the ultrasonic probe 15. In a properly tuned ultrasonic probe 15, the transverse anti-nodes 42 will be found at a position exactly one-half of the distance between the transverse nodes 40 located adjacent to each side of the transverse anti-nodes 42.

As a consequence of the transverse vibration of the ultrasonic probe 15, the occlusion destroying effects of the ultrasonic medical device 11 are not limited to those regions of the probe 15 that may come into contact with the occlusion 16. Rather, as the ultrasonic probe 15 is swept through an area of the occlusion 16, preferably in a windshield-wiper fashion, the occlusion 16 is removed in all areas adjacent to the plurality of transverse anti-nodes 42 being produced along the portion of the longitudinal axis of the ultrasonic probe 15. The preshaped segment 43 of the ultrasonic probe 15 provides a greater active area between the ultrasonic probe 15 and the occlusion 16 by maximizing the radial span of the ultrasonic probe 15 within the vasculature 44. As such, the ultrasonic probe 15 of the present invention produces a greater occlusion destroying effect. The extent of a cavitational energy produced by the ultrasonic probe 15 is such that the cavitational energy extends radially outward from the longitudinal axis of the ultrasonic probe 15 at the transverse anti-nodes 42 along the longitudinal axis of the ultrasonic probe 15. In this way, actual treatment time using the transverse mode ultrasonic medical device 11 according to the present invention is greatly reduced as compared to methods disclosed in the prior art that primarily utilize longitudinal vibration (along the axis of the ultrasonic probe) for treatment of the occlusion. Utilizing longitudinal vibration limits treatment to the tip of the probe in prior art devices.

By eliminating the axial motion of the ultrasonic probe 15 and allowing the transverse vibrations only, the active ultrasonic probe 15 can cause fragmentation of large areas of the occlusion 16 that span the entire length of the active portion of the ultrasonic probe 15 due to generation of multiple cavitational transverse anti-nodes 42 along the longitudinal axis of the ultrasonic probe 15 not parallel to the longitudinal axis of the ultrasonic probe 15. Because substantially larger affected areas can be denuded of the occlusion 16 in a short time, actual treatment time using the transverse mode ultrasonic medical device 11 according to the present invention is greatly reduced as compared to methods using prior art probes that primarily utilize longitudinal vibration (along the axis of the probe). In addition, the preshaped segment 43 of the ultrasonic probe 15 of the present invention allows for a greater amount of the transverse anti-nodes 42 to come into communication with the occlusion 16. A novel feature of the present invention is the ability to utilize ultrasonic probes 15 of extremely small diameter compared to prior art probes, without loss of efficiency, because the occlusion fragmentation process is not dependent on the area of the probe tip 9. Highly flexible ultrasonic probes 15 can therefore be designed to mimic device shapes that enable facile insertion into occlusion spaces or extremely narrow interstices that contain the occlusion 16. Another advantage provided by the present invention is the ability to rapidly remove the occlusion 16 from large areas within cylindrical or tubular surfaces. The preshaped segment 43 of the ultrasonic probe 15 allows for an increased surface area in communication with the occlusion 16, resulting in a shorter treatment time when compared to a probe that is approximately straight along the longitudinal axis. The preshaped segment 43 of the ultrasonic probe 15 focuses the delivery of the transverse ultrasonic energy to the occlusion 16, providing a focused occlusion destroying effect of the ultrasonic probe 15.

A significant advantage of the present invention is that the ultrasonic medical device 11 physically destroys and removes the occlusion 16 (especially adipose or other high water content tissue) through the mechanism of non-thermal cavitation. In a preferred embodiment of the present invention, the occlusion 16 comprises a biological material. In a preferred embodiment of the present invention, the occlusion 16 is a vascular occlusion 16. Cavitation is a process in which small voids are formed in a surrounding fluid through the rapid motion of the ultrasonic probe 15 and the voids are subsequently forced to compress. The compression of the voids creates a wave of acoustic energy which acts to dissolve the matrix binding together the occlusion 16, while having no damaging effects on healthy tissue. The ultrasonic energy source 99 provides a low power electric signal of approximately 2 watts to the transducer, which then transforms the electric signal into acoustic energy. The ultrasonic probe 15 emits low power acoustic energy of approximately 2 watts that resolves the occlusion 43 while not affecting the native vessel. Longitudinal motion created within the transducer is converted into a standing transverse wave along the portion of the longitudinal axis of the ultrasonic probe 15, which generates acoustic energy in the surrounding medium through cavitation. The acoustic energy dissolves the matrix of the occlusion 16.

The ultrasonic energy produced by the ultrasonic probe 15 is in the form of very intense, acoustic vibrations that result in physical reactions in the water molecules within a body tissue or surrounding fluids in proximity to the ultrasonic probe 15. These reactions ultimately result in a process called “cavitation,” which can be thought of as a form of cold (i.e., non-thermal) boiling of the water in the body tissue, such that microscopic voids are rapidly created and destroyed in the water creating cavities in their wake. As surrounding water molecules rush in to fill the cavity created by the collapsed voids, they collide with each other with great force. Cavitation results in shock waves running outward from the collapsed voids which can wear away or destroy material such as surrounding tissue in the vicinity of the ultrasonic probe 15.

The removal of the occlusion 16 by cavitation provides the ability to remove large volumes of the occlusion 16 with the small diameter ultrasonic probe 15, while not affecting healthy tissue. The preshaped segment 43 of the ultrasonic probe 15 expands the treatment area of the ultrasonic probe 15 to remove the occlusion 16. The use of cavitation as the mechanism for destroying the occlusion 16 allows the present invention to destroy and remove the occlusion 16 within a range of temperatures of about ±7° C. from normal body temperature. Therefore, complications attendant with the use of thermal destruction or necrosis, such as swelling or edema, as well as loss of elasticity are avoided.

The number of transverse nodes 40 and transverse anti-nodes 42 occurring along the longitudinal axis of the ultrasonic probe 15 is modulated by changing the frequency of energy supplied by the ultrasonic energy source 99. The exact frequency, however, is not critical and for the ultrasonic probe 15, the ultrasonic energy source 99 run at, for example, about 20 kHz is generally sufficient to create an effective number of occlusion destroying transverse anti-nodes 42 along the longitudinal axis of the ultrasonic probe 15. The mechanical vibration of the ultrasonic probe 15 at the low frequency of about 20 kHz provides a safer condition for healthy tissue. Since tissue absorbs low frequency sound less than high frequency sound, the present invention prevents damage to healthy tissue. Those skilled in the art understand it is possible to adjust the dimensions of the ultrasonic probe 15, including diameter, length and distance to the ultrasonic energy source 99, in order to affect the number and spacing of the transverse nodes 40 and transverse anti-nodes 42 along a portion of the longitudinal axis of the ultrasonic probe 15.

The present invention allows the use of ultrasonic energy to be applied to occlusions 16 selectively, because the ultrasonic probe 15 conducts energy across a frequency range from about 20 kHz through about 80 kHz. The amount of ultrasonic energy to be applied to a particular treatment site is a function of the amplitude and frequency of vibration of the ultrasonic probe 15. In general, the amplitude or throw rate of the energy is in the range of about 25 microns to about 250 microns, and the frequency in the range of about 20 kHz to about 80 kHz. In a preferred embodiment of the present invention, the frequency of ultrasonic energy is from about 20 kHz to about 35 kHz. Frequencies in this range are specifically destructive of occlusions 16 including, but not limited to, hydrated (water-laden) tissues such as endothelial tissues, while substantially ineffective toward high-collagen connective tissue, or other fibrous tissues including, but not limited to, vascular tissues, epidermal, or muscle tissues.

The amount of cavitation energy to be applied to a particular site requiring treatment is a function of the amplitude and frequency of vibration of the ultrasonic probe 15, the longitudinal length of the ultrasonic probe 15, the geometry at the distal end 24 of the ultrasonic probe 15, the proximity of the ultrasonic probe 15 to the occlusion 16, and the degree to which the length of the ultrasonic probe 15 is exposed to the occlusion 16. Reducing the amount of energy from the ultrasonic energy source 99 can reduce the amount of damage to healthy tissue.

The ultrasonic probe 15 is inserted into a vasculature 44 of the body and may be disposed of after use. In a preferred embodiment of the present invention, the ultrasonic probe 15 is for a single use and on a single patient. In a preferred embodiment of the present invention, the ultrasonic probe 15 is disposable. In another embodiment of the present invention, the ultrasonic probe 15 can be used multiple times.

The present invention provides a method of expanding a treatment area of the ultrasonic probe 15 to ablate the occlusion 16 in the vasculature 44 of the body. The ultrasonic probe 15 with the preshaped segment 43 is inserted into the catheter 36. A section of the longitudinal axis of the ultrasonic probe 15 is advanced beyond the distal end 37 of the catheter 36 and positioned in proximity to the site of the occlusion 16. Once the ultrasonic probe 15 is positioned within the region of the occlusion 16, the ultrasonic energy source 99 is activated, producing a plurality of transverse nodes 40 and transverse anti-nodes 42 along the longitudinal axis of the ultrasonic probe 15 to ablate the occlusion 16. In one embodiment of the present invention, the section of the longitudinal axis of the ultrasonic probe 15 is advanced to the site of the occlusion 16 by pushing the section of the longitudinal axis of the ultrasonic probe 15 past the distal end 37 of the catheter 36. In another embodiment of the present invention, the section of the longitudinal axis of the ultrasonic probe 15 is advanced to the site of the occlusion 16 by pulling back on the catheter 36 such that the section of the longitudinal axis of the ultrasonic probe 15 is outside of an interior of the catheter 36.

In an embodiment of the present invention shown in FIG. 2, the effective radial span from the preshaped segment 43 has the first outermost point 47 and the second outermost point 49. In an embodiment of the present invention, the radial span of the first outermost point 47 and the second outermost point 49 of the preshaped segment 43 is larger than an inside radius of the catheter 36. Those skilled in the art will recognize that the first outermost portion and the second outermost portion of the preshaped segment 43 at the distal end of the ultrasonic probe can create a radial span that is larger than or small than the inside diameter of the catheter and be within the spirit and scope of the present invention. As such, the present invention treats an area that is larger than is possible with prior art probes.

The present invention also provides a method of increasing a surface area of the ultrasonic probe 15 in communication with the occlusion 16. The ultrasonic probe 15 with the preshaped segment 43 is inserted into the catheter 36 and the section of the longitudinal axis of the ultrasonic probe 15 is advanced past the distal end 37 of the catheter 36. The transducer transmits ultrasonic energy received from the ultrasonic energy source 99 to the ultrasonic probe 15.

The preshaped segment 43 of the ultrasonic probe 15 can be compressed within the interior of the catheter 36 such that when the preshaped segment 43 of the ultrasonic probe 15 is moved past the distal end 37 of the catheter 36, the preshaped segment 43 releases from the interior of the catheter 36 and can spring out of the distal end 37 of the catheter 36. The larger effective area span with the preshaped segment 43 of the ultrasonic probe 15 allows for a larger active area with the occlusion 16 and expands a treatment area of the ultrasonic probe 15. The increased surface area from the preshaped segment 43 of the ultrasonic probe 15 in the region of the occlusion 16 allows for a low power and reduced treatment time for the more complete ablation of the occlusion 16. Lower power levels and reduced treatment time allow for less damage to healthy tissue of the vasculature 44.

The present invention provides a method of expanding the treatment area of the ultrasonic probe 15 to ablate an occlusion 16 in the vasculature 44. The ultrasonic probe 15 with the preshaped segment 43 along the longitudinal axis is inserted into the catheter 36 and the preshaped segment 43 is advanced beyond a distal end 37 of the catheter 36. An ultrasonic energy source 99 is activated to provide the ultrasonic energy to the ultrasonic probe 15 to ablate the occlusion 16.

The present invention provides a method of increasing the surface area of the ultrasonic probe 15 in communication with the occlusion 16 to ablate the occlusion 16. The ultrasonic probe 15 with the preshaped segment 43 is advanced to the site of the occlusion 16 and the transducer transmits the transverse ultrasonic energy from the ultrasonic energy source 99 to ultrasonic probe 15. In one embodiment of the present invention, the ultrasonic probe 15 is pushed back and forth through the occlusion 16. In another embodiment of the present invention, the ultrasonic probe 15 is swept along the occlusion 16. In another embodiment of the present invention, the ultrasonic probe 15 is twisted through the occlusion 16. In another embodiment of the present invention, the ultrasonic probe 15 is rotated through the occlusion 16. Rotating, twisting, pushing back and forth or sweeping the ultrasonic probe 15 through the occlusion 16 allows for the increased radial span from the preshaped segment 43 to engage the occlusion 16 and expand the treatment area of the ultrasonic probe 15 by having a greater surface area of the ultrasonic probe 15 in communication with the occlusion 16.

The present invention provides a method of increasing the surface area of the ultrasonic probe 15 in communication with the occlusion 16. The ultrasonic probe 15 with the preshaped segment 43 along the longitudinal axis is advanced to the site of the occlusion 16. The preshaped segment 43 of the ultrasonic probe 15 is placed in communication with the occlusion 16 and an ultrasonic energy source 99 is activated to vibrate the longitudinal axis of the ultrasonic probe 15 in a transverse direction to ablate the occlusion 16.

The combination of the ultrasonic energy and the increased surface area of the preshaped segment 43 of the ultrasonic probe 15 engaging the occlusion 16, allows for the more complete removal of the occlusion 16 and for lower power out of the ultrasonic energy source 99. As the ultrasonic probe 15 is moved through the occlusion 16, either by axial motion, twisting or rotation, the cavitational effects are enhanced by the preshaped segment 43.

The present invention provides an apparatus and method for increasing the surface area of the ultrasonic probe 15 in communication with the occlusion 16 to remove an occlusion 16 within a vasculature 44. The preshaped segment 43 of the ultrasonic probe 15 maximizes a radial span of the ultrasonic probe 15 and expands a treatment area of the ultrasonic probe 15. The preshaped segment 43 of the ultrasonic probe 15 is adapted to engage the vasculature 44 and adapts to the contour of the vasculature 44. The maximized radial span from the preshaped segment 43 of the ultrasonic probe 15 allows for a larger active area of the ultrasonic probe 15 in communication with the occlusion 16 and for a lower power level and reduced treatment time in the ablation of the occlusion 16. The present invention provides an apparatus and a method for an ultrasonic medical device 11 with an ultrasonic probe 15 having a preshaped segment 43 that is simple, user-friendly, reliable and cost effective.

All patents, patent applications, and published references cited herein are hereby incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. An ultrasonic medical device comprising:
an ultrasonic probe having a proximal end, a distal end and a longitudinal axis therebetween; and
a preshaped segment along the longitudinal axis of the ultrasonic probe, wherein the preshaped segment increases a surface area of the ultrasonic probe in communication with a biological material.

2. The device of claim 1 wherein the preshaped segment is located at the distal end of the ultrasonic probe.

3. The device of claim 1 wherein the preshaped segment is curved.

4. The device of claim 1 wherein the preshaped segment extends from the longitudinal axis of the ultrasonic probe at an angle.

5. The device of claim 1 wherein a configuration of the preshaped segment is selected from a group consisting of a sinusoidal shape, a S-shape, a coiled spring, a corkscrew, a hook and similar shapes.

6. The device of claim 1 wherein the preshaped segment provides a large active area for ablation of the biological material.

7. The device of claim 1 wherein the preshaped segment is adapted to engage a vasculature.

8. The device of claim 1 wherein the preshaped segment maximizes a radial span of the ultrasonic probe within a vasculature.

9. The device of claim 1 wherein the preshaped segment expands a treatment area of the ultrasonic probe.

10. The device of claim 1 wherein the preshaped segment focuses a biological material destroying effect of the ultrasonic probe.

11. The device of claim 1 wherein the preshaped segment adapts to a contour of a vasculature.

12. The device of claim 1 wherein the preshaped segment allows the ultrasonic probe to be moved through a vasculature of a body without damaging the vasculature.

13. The device of claim 1 wherein the preshaped segment engages the biological material.

14. The device of claim 1 wherein the ultrasonic probe is a wire.

15. The device of claim 1 further comprising a plurality of preshaped segments along the longitudinal axis of the ultrasonic probe.

16. An ultrasonic medical device for ablating an occlusion comprising:
an elongated flexible probe having a preshaped segment along a longitudinal axis that maximizes a radial span of the elongated flexible probe within a vasculature; and
a catheter having a proximal end and a distal end, the catheter surrounding a length of the longitudinal axis of the elongated flexible probe,
wherein the elongated flexible probe supports a transverse ultrasonic vibration along a portion of the longitudinal axis of the elongated flexible probe to ablate the occlusion.

17. The device of claim 16 wherein the preshaped segment focuses a delivery of a transverse ultrasonic energy to the occlusion.

18. The device of claim 16 wherein the preshaped segment increases a surface area of the elongated flexible probe in communication with the occlusion.

19. The device of claim 16 wherein the preshaped segment is adapted to engage the vasculature.

20. The device of claim 16 wherein the preshaped segment expands a treatment area of the elongated flexible probe.

21. The device of claim 16 wherein the preshaped segment focuses an occlusion destroying effect of the elongated flexible probe.

22. The device of claim 16 wherein the preshaped segment adapts to a contour of the vasculature.

23. The device of claim 16 wherein the preshaped segment is located at a distal end of the elongated flexible probe.

24. The device of claim 16 wherein the transverse ultrasonic vibration of the elongated flexible probe produces a plurality of transverse nodes and transverse anti-nodes along the portion of the longitudinal axis of the elongated flexible probe.

25. The device of claim 24 wherein the plurality of transverse anti-nodes are points of a maximum transverse energy along the portion of the longitudinal axis of the elongated flexible probe.

26. The device of claim 24 wherein the plurality of transverse anti-nodes cause a cavitation in a medium in communication with the elongated flexible probe in a direction not parallel to the longitudinal axis of the elongated flexible probe.

27. The device of claim 24 wherein more than one of the plurality of transverse anti-nodes are in communication with the occlusion.

28. The device of claim 16 wherein the occlusion comprises a biological material.

29. The device of claim 16 further comprising a plurality of preshaped segments along the longitudinal axis of the elongated flexible probe.

30. A method of expanding a treatment area of an ultrasonic probe to ablate a biological material in a vasculature of a body comprising:
inserting the ultrasonic probe having a preshaped segment along a longitudinal axis of the ultrasonic probe into a catheter;
advancing the preshaped segment of the ultrasonic probe beyond a distal end of the catheter; and
activating an ultrasonic energy source to provide an ultrasonic energy to the ultrasonic probe to ablate the biological material.

31. The method of claim 30 wherein the preshaped segment is located at a distal end of the ultrasonic probe.

32. The method of claim 30 wherein the preshaped segment is curved.

33. The method of claim 30 wherein the preshaped segment extends from the longitudinal axis of the ultrasonic probe at an angle.

34. The device of claim 30 wherein a configuration of the preshaped segment is selected from a group consisting of a sinusoidal shape, a S-shape, a coiled spring, a corkscrew, a hook and similar shapes.

35. The method of claim 30 wherein the ultrasonic probe is advanced beyond the distal end of the catheter by pushing the ultrasonic probe through the catheter.

36. The method of claim 30 wherein the ultrasonic probe is advanced beyond the distal end of the catheter by pulling back on the catheter.

37. The method of claim 30 further comprising moving the ultrasonic probe back and forth along the biological material.

38. The method of claim 30 further comprising sweeping the ultrasonic probe along the biological material.

39. The method of claim 30 further comprising rotating the ultrasonic probe along the biological material.

40. The method of claim 30 further comprising twisting the ultrasonic probe along the biological material.

41. The method of claim 30 wherein the preshaped segment allows the ultrasonic probe to be moved through the vasculature without damaging the vasculature.

42. The method of claim 30 wherein the preshaped segment engages the biological material.

43. The method of claim 30 wherein the preshaped segment is adapted to engage the vasculature.

44. The method of claim 30 wherein the preshaped segment maximizes a radial span of the ultrasonic probe in the vasculature.

45. The method of claim 30 wherein the preshaped segment focuses a biological material destroying effect of the ultrasonic probe.

46. The method of claim 30 wherein the preshaped segment adapts to a contour of the vasculature.

47. The method of claim 30 wherein a length and a cross section of the ultrasonic probe are sized to support a transverse ultrasonic vibration with a plurality of transverse nodes and transverse anti-nodes along a portion of the longitudinal axis of the ultrasonic probe wherein more than one of the plurality of transverse anti-nodes are in communication with the biological material.

48. The method of claim 30 wherein the preshaped segment increases a surface area of the ultrasonic probe in communication with the biological material.

49. The method of claim 30 wherein the preshaped segment focuses a delivery of a transverse ultrasonic energy to the biological material.

50. The method of claim 30 wherein the ultrasonic probe is a wire.

51. The method of claim 30 wherein the ultrasonic probe comprises a plurality of preshaped segments along the longitudinal axis of the ultrasonic probe.

52. A method of increasing a surface area of a flexible ultrasonic probe in communication with an occlusion comprising:
advancing the flexible ultrasonic probe with a preshaped segment along a longitudinal axis to a site of the occlusion;
placing the preshaped segment of the flexible ultrasonic probe in communication with the occlusion; and
activating an ultrasonic energy source to vibrate the longitudinal axis of the flexible ultrasonic probe in a transverse direction to ablate the occlusion.

53. The method of claim 52 further comprising moving the flexible ultrasonic probe back and forth along the occlusion.

54. The method of claim 52 further comprising sweeping the flexible ultrasonic probe along the occlusion.

55. The method of claim 52 further comprising rotating the flexible ultrasonic probe along the occlusion.

56. The method of claim 52 further comprising twisting the flexible ultrasonic probe along the occlusion.

57. The method of claim 52 wherein a length and a cross section of the flexible ultrasonic probe are sized to support a transverse ultrasonic vibration with a plurality of transverse nodes and transverse anti-nodes along a portion of the longitudinal axis of the flexible ultrasonic probe wherein more than one of the plurality of transverse anti-nodes are in communication with the occlusion.

58. The method of claim 57 wherein the plurality of transverse anti-nodes cause a cavitation in a medium in communication with the flexible ultrasonic probe in a direction not parallel to the longitudinal axis of the flexible ultrasonic probe.

59. The method of claim 52 wherein the preshaped segment maximizes a radial span of the flexible ultrasonic probe within a vasculature.

60. The method of claim 52 wherein the preshaped segment focuses an occlusion destroying effect of the flexible ultrasonic probe.

61. The method of claim 52 wherein the flexible ultrasonic probe is for a single use on a single patient.

62. The method of claim 52 wherein the flexible ultrasonic probe is disposable.

63. The method of claim 52 wherein the occlusion comprises a biological material.

64. The method of claim 52 wherein the flexible ultrasonic probe comprises a plurality of preshaped segments along the longitudinal axis of the flexible ultrasonic probe.
